SEQUENCE LISTING (4 sequences)

SEQ ID NO: 1 - DNA, 615 bp, infectious hypodermal hematopoietic necrosis virus
atggatctttctttcactctttcggtcgtgtcggccatcctcgccatcactgctgtgattgctgtatttattgtgatttttaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 2 - protein, 204 aa, white spot syndrome virus
MDLSFTLSVVSAILAITAVIAVFIVIFRYHNTVTKTIETHTDNIETNMDENLRIPVTAEVGSGYFKMTDVSFDSDTLGKIKIRNGKSDAQMKEEDADLVITPVEGRALEVTVGQNLTFEGTFKVWNNTSRKINITGMQMVPKINPSKAFVGSSNTSSFTPVSIDEDEVGTFVCGTTFGAPIAATAGGNLFDMYVHVTYSGTETE

SEQ ID NO: 3 - DNA, 990 bp, infectious hypodermal hematopoietic necrosis virus
atgtgcgccgattcaacaagagcaagcccaaggaaaagatccaggagggatgcacataatgaagacgaagaacacgccgaaggatcaagtggaccagacccacacagatgtctacaattcaatactggagactcaatacatattactttccaaacaagaagatacttcgaattcgacgctgccaatgatggaaacttcgacggaaaaaatttatactgcctcccactacattggatgaacttatatctctatggtctaaagagcagcgacagttcagcaacagaaacacaacgatataagatggtaaaatcaatgatgaagacctacggatggaaagtacataaagcaggcgtagtgatgcactcgatggtaccccttatgaaagacttaaaagtatcaggaggcacatcatttgagactctcacatttacagacaccccatatttagaaatatttaaggatactactggactacataatcaactatcaactaaggaagccgacgtaacattggcaaaatggatacaaaatccccaacttgtgaccgtacaatcaacagcagcaaactatgaagacccaatccaacaatttggattcatggaacaaatgcgaaccggtgacagaaaagcctatacaatccatggtgacactagaaattggtatggcggagaaataccaacaaccggacccaccttcatcccaaaatggggtggtcaattaaaatgggacaaaccatcccttggaaacctagtctacccagcagaccaccatacaaacgactggcaacagatcttcatgagaatgtcaccaatcaaaggaccaaatggagacgaacttaaacttggctgcagagtacaagccgacttcttcctacacctagaagtacgactcccaccacaaggatgtgtcgcaagtttggggatgttacaatatcttcacgcaccatgtactggacaacttaacaaatgttatattatgcatactaactaa

SEQ ID NO: 4 - protein, 329 aa, infectious hypodermal hematopoietic necrosis virus
MCADSTRASPRKRSRRDAHNEDEEHAEGSSGPDPHRCLQFNTGDSIHITFQTRRYFEFDAANDGNFDGKNLYCLPLHWMNLYLYGLKSSDSSATETQRYKMVKSMMKTYGWKVHKAGVVMHSMVPLMKDLKVSGGTSFETLTFTDTPYLEIFKDTTGLHNQLSTKEADVTLAKWIQNPQLVTVQSTAANYEDPIQQFGFMEQMRTGDRKAYTIHGDTRNWYGGEIPTTGPTFIPKWGGQLKWDKPSLGNLVYPADHHTNDWQQIFMRMSPIKGPNGDELKLGCRVQADFFLHLEVRLPPQGCVASLGMLQYLHAPCTGQLNKCYIMHTN

(19) United States
(12) Patent Application Publication
(10) Pub. No.: US 2005/0158326 A1
(43) Pub. Date: Jul. 21, 2005

(54) Compositions for reducing virus infection rate in aquatic crustaceans and applications thereof

(76) Inventors: Chin-Yu Chen, Taipei (TW); Tai-Hsin Yang, Taipei (TW); Chan-Yen Tsai, Taipei (TW); Woan-Jiun Swei, Taipei (TW); Ming-Chuan Chang, Taipei (TW)

Correspondence Address: RABIN & Berdo, PC, 1101 14TH STREET, NW, SUITE 500, WASHINGTON, DC, 20005, US

(21) Appl. No.: 11/014,968

(22) Filed: Dec. 20, 2004

Related U.S. Application Data
(60) Provisional application No. 60/532,646, filed on Dec. 24, 2003.

(51) Int. Cl. C12Q 1/70; A61K 39/42

(52) U.S. Cl. 424/159.1

(57) ABSTRACT

Disclosed is a composition for reducing virus infection rates in crustaceans, which can be applied in prevention and/or treatment of viral infection in crustaceans, and therefore improves the survival rate. The composition comprises at least one of the antibodies that can bind specifically to virus, and the antibodies are selected from the group consisting of monoclonal antibody, phage display antibody and antibody produced by a recombinant organism. The monoclonal antibodies can be produced in a large scale from hybridoma cells with a bioreactor or by injecting into the abdominal cavities of mice. Alternatively, two other highly specific antibodies can be produced from phage clones and recombinant organisms. The composition can be used in the forms of therapeutic medicines, nutritious or feeding supplements in addition to feeds. Also, the composition can be used in an aqueous solution to expose the crustaceans to fulfill the needs of treatment and/or prevention of viral infection in crustaceans.

### CROSS-REFERENCE TO RELATED APPLICATION This application claims the benefit of U.S. Provisional application No. 60/532,646, filed Dec. 24, 2003.

### BACKGROUND OF THE INVENTION

1. Field of the Invention

The present invention relates to aquaculture management, especially relates to a composition and method for reducing virus infection rates in crustaceans, particularly shrimps and crabs, which can be applied in prevention and/or treatment of viral infection.

2. The Prior Arts

The progress on artificial breeding technology, the demand for market and high-profit have made aquaculture production an important industry. According to studies of Food Agriculture Organism, (FAO), due to the declines of fisheries in global catches and the world's quickly growing population, farming seafood offers a solution to meet the growing demand for seafood that catching fish cannot provide. In 2001, the total worldwide shrimp production was 1.27 million tons, wherein the giant black tiger shrimp Penaeus monodon contributes a major share of 0.61 million tons.

However, because of environmental contamination in aquaculture farms, and some uncontrollable microbial infection, especially viral infection, viral spread is rapidly and uncontrollable. Viral infections cause the problems of no effective treatment and dramatic reduction of production to global hatchery managers. Therefore, effectively control of the viral infection becomes an important and prompt issue.

In general, aquaculture producers expect to cultivate animals in high density to obtain high production yield from the same unit based on economical consideration. However, serious infection of pathogens and environmental contamination result in devastating losses to aquaculture farmers. For example, shrimps, crabs and other crustaceans are susceptible to some same viral infections. Once virus infection occurs, spread between crustaceans is rapid (for example, virus infected crabs would transmit to shrimps) and results in a high mortality rate.

The most popular seafood-shrimp is taken as an example; farmers collect the eggs after the female shrimps spawning, chlorinate these eggs and wash with clean water. The fertilized eggs in hatchery pond will hatch to nauplii and enter five stages of growth: zoeal stages, mysis stages, postlarval stages, juvenile stages and adult stages. Once the cultured shrimps get ill, the production will be forced to cease, which will cause the most serious economic damage in shrimp farmers.

There are about 20 viruses known to be highly pathogenic to shrimps, for example: infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), white spot syndrome virus (WSSV) and so on. However, these diseases cannot be treated by the known medication such as copper sulfate, potassium permanganate, formalin, malachite green, oxytetracycline, iodoform I-500, furyl drug, or sulfa drugs. In addition, there are problems of drug residue and drug resistance with the abovementioned drugs. And the infected shrimps are frequently detected with two or more than two viruses, the so-called “mixed infection”. This situation makes the virus control of shrimps even more complicate and difficult (Diseases of Aquatic Organisms, 48, p 233-236, 2002; Fish Pathology, 35(1), 1-10, 2000; Fish Pathology 24(2), p 89-100, 1989).

To solve the problems of virus infection in crustaceans, many people have suggested methods to increase the resistance of crustaceans toward virus, in order to increase the survival rate of aquaculture. For example, Manohar et al. have suggested in U.S. Pat. No. 6,440,466 one composition containing effective amount of extract obtained from the plants for the management of viral and bacterial diseases in aquatic animals. In another case, Laramore et al. disclosed one composition and method for inducing tolerance of aquatic animals to virus infections in U.S. Pat. No. 6,705,556. The US patent provided a tolerine composition based on inactivated viral particles of White Spot Syndrome Virus by exposing larval shrimps to the tolerine composition for inducing tolerance toward viral infection in larval shrimps. However, the induction is not effective because the immune systems of larval shrimps are not mature enough to protect themselves, and the induction needs time to insure the therapeutic effects. People also applied polyclonal antibodies against the major envelope protein of virus to obtain direct therapeutic effects by enhancing the anti-viral abilities of aquatic animals. For example, Van Hulten et al. disclosed specific antibodies against white spot syndrome virus to prevent the infection of this virus. Their method was carried out by intramuscular injection of the polyclonal antibodies into shrimp bodies (Van Hulten et al. White spot syndrome virus envelop protein VP28 is involved in the systemic infection of shrimp. 2001; Virology 285, 228-233). Handling of intramuscular injection into shrimp bodies is extremely time-consuming and laborious. Therefore, it is not suitable for cultivation breeding. In addition, that reference did not mention how to treat or prevent the viral infection with high prevalence rate toward larval shrimps. On the other hand, patent application numbered PCT WO 03/070258, Lee et al. disclosed an anti-WSSV antibody produced on large scale in egg yolk immunoglobulin Y (IgY). The hens or ducks were immunized with WSSV antigens such as inactivated virus, dead virus or viral proteins, to generate antibodies against WSSV in egg yolks. The antibodies needed were purified from eggs, which is not stable in amount and difficult in purification. Also, Hulten et al. described several WSSV specific viral proteins (VP19, VP24, VP26 and VP28) in patent application number WO 01/09340. These viral proteins could be applied in recognition of such viral proteins, also in generating antibodies and vaccines. In addition, said application also disclosed polyclonal antibody production by injection of these viral proteins into rabbits as well as immunization of shrimps with these polyclonal antibodies to enhance the tolerance toward viral infection. However, people who are skilled in the art know that polyclonal antibodies are not as effective as monoclonal antibodies in anti-viral effects, and polyclonal antibodies will not only react with target virus but also induce unnecessary immune reaction.

Therefore, the present invention is devoted to provide a composition and an application way to reduce virus infection rate in crustaceans.

### SUMMARY OF THE INVENTION

A primary object of the present invention is to provide a composition for prevention and/or treatment of viral infection in order to control virus infection rates in crustaceans. The composition comprises at least one of the antibodies selected from the group consisting of monoclonal antibody, phage display antibody and antibody produced by a recombinant organism, which can bind specifically to virus. Said composition can be applied in preparation of medicine composition, nutrition composition, feed additives or feed composition.

Another object of the present invention is to effectively produce monoclonal antibodies in large quantities from hybridoma cells with a bioreactor or by injecting into the abdominal cavities of mice.

Particularly, the above-mentioned crustaceans are shrimps and crabs.

On the other hand, the present invention also provides a method for prevention and/or treatment of viral infection, which comprises the steps of mixing a composition comprising at least one of the viral-specific antibodies with a medium, and administrating the mixture to shrimps in various ways according to the different cultivated stages. Said antibody may be monoclonal antibody, phage display antibody or antibody produced by a recombinant organism, and said medium may be breeding water or feed. For example:
(a) Soak gravid shrimps in a solution containing said composition;
(b) Soak fertilized eggs in a solution containing said composition;
(c) Soak nauplii in a solution containing said composition;
(d) Soak zoea in a solution containing said composition, feed zoea with said feeding composition;
(e) Soak mysis shrimps in a solution containing said composition, feed mysis shrimps with said feeding composition;
(f) Soak postlarvae shrimps in a solution containing said composition, feed postlarval shrimps with said feeding composition;
(g) Deliver a package of said feeding composition to juvenile shrimps;
(h) Deliver a package of said feeding composition to mature shrimps.

The monoclonal antibodies provided in the present invention is preferably to be monoclonal antibodies against crustaceans virus, which is selected from the group consisting of infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), and white spot syndrome virus (WSSV). The composition of the present invention preferably comprises at least one of the monoclonal antibodies against the abovementioned virus, most preferably comprises at least two of the monoclonal antibodies, in order to enhance the ability of treatment and/or prevention of viral infection. When two or more than two of the monoclonal antibodies are included in the composition, they can be applied to resist the same or different virus. The different viral antibodies are preferred to effectively resist various mixed virus infection and increase the survival rate of shrimps.

Another object of the present invention is to provide a production method of composition for prevention and/or treatment of viral infection in order to control virus infection rates in crustaceans. Said composition comprises at least one of the viral-specific monoclonal antibodies, and said monoclonal antibodies may be produced in large quantities after collecting culture supernatant of hybridoma from a bioreactor. When large-scale production of monoclonal antibodies is carried out in a large bioreactor, the preferred volume of the bioreactor is at least one liter. Monoclonal antibodies can also be produced by injecting the hybridoma into the abdominal cavities of mice to induce the formation of tumor, and highly concentrated monoclonal antibodies are obtained from ascitic fluid of mice after fine needle aspiration. Usually there are 15 ml of ascitic fluid per mouse. High levels of monoclonal antibodies can be obtained from the methods disclosed in the present invention economically and effectively.

Alternatively, highly specific phage display antibody or antibody produced by a recombinant organism is also available for said composition. Phage display system is a useful system to screen out an antibody or antibody fragment specific for the antigen of interest by displaying the functional binding sites on the surface of M13 filamentous phage (Burton & Barbas. Human antibodies from combinatorial libraries. 1994; Adv. Immunol., 57, 191-280). Phage display system can screen out phage clones displaying an antibody or antibody fragment specific to the virus and nucleotides encoding an antibody or antibody fragment specific to the virus. The phage clones displaying desired antibody can be cultivated and enriched in a host organism system. And phage display antibodies can be obtained and recovered from the host organism system. In another way, a nucleotide selected with the phage display system, which encodes a desired antibody or antibody fragment, can be transformed into a host organism system to obtain a recombinant organism. The recombinant organism may be a bacterial or yeast cell. The recombinant organism can be used to produce antibodies specific to the virus.

The present invention is further explained in the following embodiment illustration and examples. The present invention disclosed above is not limited by these examples. The present invention may be altered or modified and all such variations are within the scope and spirit of the present invention.

### BRIEF DESCRIPTION OF THE DRAWINGS

People who skilled in the art will understand the invention with the related drawings in connection with the detailed description of the present invention which described briefly as follows, in which:

FIG. 1 shows the PCR amplification product from IHHNV. The DNA sequence of VP37 gene from IHHNV is 1038 bp in length.

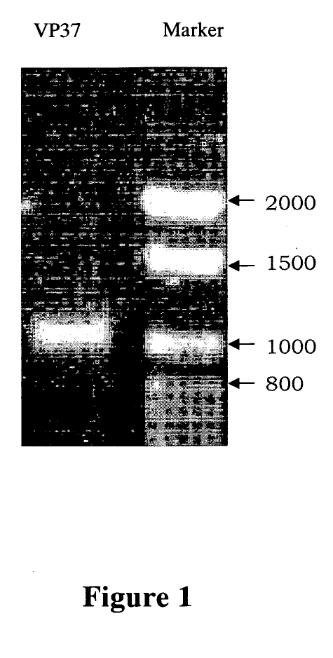

FIG. 2 shows the SDS-PAGE analysis of E. coli BL21 pLysS harboring VP37 expression plasmid pET28a-VP37. Lane 1: crude lysate of transformed E. coli before IPTG induction; Lane 2: crude lysate of transformed E. coli after IPTG induction. Arrow indicates the position of recombinant VP37 after hyper expression.

FIG. 3 shows the Western blot analysis of anti-WSSV VP28 antibodies against WSSV virus particles and VP28 recombinant protein. Lane 1: purified WSSV virus particles after CsCl discontinuous gradient ultracentrifugation; Lane 2: VP28 recombinant protein; Lane 3: protein markers. The antibody is diluted in the ratio of 1:2000.

FIG. 4 shows the survival rates of shrimps with different treatments. Block diamond represents monoclonal antibody; Block triangle represents infected shrimp extracts and WSSV monoclonal antibody; Block square represents infected shrimp extracts.

FIG. 5 shows the survival rates of large-size shrimps (3.92±10.28 cm in length, and on average 0.45 g in weight) with different treatments. Block diamond represents monoclonal antibody; Block triangle represents infected shrimp extracts and WSSV monoclonal antibody; Block square represents infected shrimp extracts.

FIG. 6 shows the survival rates of medium-size shrimps (3.32±0.32 cm in length, and on average 0.25 g in weight) with different treatments. Block diamond represents monoclonal antibody; Block triangle represents infected shrimp extracts and WSSV monoclonal antibody; Block square represents infected shrimp extracts.

FIG. 7 shows the survival rates of small-size shrimps (2.48±0.29 cm in length, and on average 0.15 g in weight) with different treatments. Block diamond represents monoclonal antibody; Block triangle represents infected shrimp extracts and WSSV monoclonal antibody; Block square represents infected shrimp extracts.

FIG. 8 shows the treatment effects of HTWC antibody to infected shrimps. Block diamond represents control group, no antibody in diet, virus infection only; Block triangle represents antibody supplemented to diet after viral infection for one day; Block square represents antibody supplemented to diet after viral infection for 3 days; Block circle represents antibody supplemented to diet after viral infection for 5 days; Star represents antibody supplemented to diet after viral infection for 7 days.

### DETAILED DESCRIPTION OF THE PREFERRED EMBODIMENT

The present invention provides a composition for prevention and/or treatment of viral infection in order to control virus infection rates in crustaceans. The composition comprises at least one of the antibodies selected from the group consisting of monoclonal antibody, phage display antibody and antibody produced by a recombinant organism, which can bind specifically to virus. Said composition can be applied in crustaceans through soaking or feeding. The present invention also provides a composition for prevention and/or treatment of viral infection economically and conveniently.

The aforementioned crustaceans in the invention preferably are cultivated in high-density aquaculture farming systems, most preferably are serious infected crustaceans on which no effective treatment is known. Said crustaceans are preferably shrimps or crabs.

The infection in the invention relates to pathogens such as prokaryotes or eukaryotes, wherein said infection is preferably related to virus, and most preferably related the group consisting of infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), and white spot syndrome virus (WSSV).

Antibodies like monoclonal antibody, phage display antibody and antibody produced by a recombinant organism according to the invention are bound specifically to the abovementioned pathogens, such as infected virus.

There is no particular limitation to the product form of the composition, as long as the antibody is not destroyed in the desired forms. For example, the composition can be medical composition, nutrition supplement, feed additive and feed composition, and not limited to such forms.

The medical composition according to the invention comprises effective dosage of at least one of the antibodies selected from the group consisting of monoclonal antibody, phage display antibody and antibody produced by a recombinant organism, which can bind specifically to virus. The term “effective dosage” refers to the dosage enough for crustaceans to resist viral infection, which is different and depends upon the species of monoclonal antibody, ways of delivery, the timing for delivery, cultivating temperature and the ages and health conditions of crustaceans.

The composition of nutrition supplement and feed additive according to the invention comprise effective dosage of at least one of the monoclonal antibodies, which can deliver with nutrition composition or feed at the same time. It can be applied in prevention and/or treatment of viral infection in crustaceans, and therefore improves the survival rates and yields. The term “effective dosage” refers to the dosage enough for crustaceans to resist viral infection, which is different and depends upon the species of monoclonal antibody, ways of delivery, the timing for delivery, cultivating temperature and the ages and health conditions of crustaceans.

On the other hand, the invention also provides a method for prevention and/or treatment of viral infection in shrimps. The composition comprises at least one of antibodies selected from the group consisting of monoclonal antibody, phage display antibody and antibody produced by a recombinant organism, which can bind specifically to virus is mixed with a medium and administrate to shrimps in specified growth stages. The medium may be breeding water or feeds, and the specified growth stages may be gravid shrimp stage, fertilized-egg stage, nauplii stage, zoeal stage, mysis stage, postlarvae stage, juvenile stage or adult shrimp stage. Examples of treatment or/and prevention of shrimp viral infection are listed below:
(1) During gravid shrimp stage, the gravid shrimps are transferred to pond containing the composition every 2-3 days for a certain period of time, and then transferred back to the breeding pond.

(2) During fertilized egg stage, the fertilized eggs are harvested after shrimp spawning for 8-10 hour. The eggs are chlorinated, rinsed with clean seawater, moved to the hatchery pond and soaked in clean seawater. The composition is added to clean seawater and poured into the hatchery pond.

(3) During nauplii stage, nauplii are usually hatched in the hatchery pond after 12-18 hour post-ovulation. It is not necessary to supply feeds since nauplii obtain enough nutrition from egg yolk during metamorphosis. Therefore, said composition according to the invention is delivered to water directly to soak nauplii.

(4) During zoeal stage, zoea starts to ingest diet. Generally, feeds comprise 3-5 μm phytoplankton, fertilized eggs of oyster and artificial plankton. At this stage, the composition of the invention can be directly added into clean seawater or mixed with feeds to be delivered to zoea.

(5) During mysis stage, feeds of mysis shrimps usually comprise phytoplankton, artificial plankton, artemia nauplii and rotifera. At this stage, the composition of the invention can be directly added into clean seawater or mixed with feeds to be delivered to mysis shrimps.

(6) During postlarvae stage, feeds of postlarvae shrimps usually comprise phytoplankton, artificial plankton, and artemia. At this stage, the composition of the invention can be directly added into clean seawater or mixed with feeds to be delivered to postlarvae shrimps.

(7) During juvenile shrimp stage, feeds of juvenile shrimps usually comprise artificial feeds, soybean powder, oyster, fish meats, and shrimp meats. At this stage, the composition of the invention can be mixed with feeds to be delivered to juvenile shrimps.

(8) During adult shrimp stage, adult shrimps will grow till being sold to market. Feeds usually comprise artificial feeds, soybean powder, oyster, fish meats, and shrimp meats. At this stage, the composition of the invention can be mixed with feeds to be delivered to adult shrimps.

All the abovementioned compositions containing antibodies can be replaced with antibodies only.

The steps described in the invention can be performed continuously or when needed. Preferably, the steps are carried out in each stage starting from fertilized eggs to adult shrimps. The method of the invention provides a convenient and effective treatment and/or prevention method, which increases survival rates and further enhances aquaculture yield of shrimps to a large extent.

Monoclonal antibodies used in the present invention can be prepared with any methods known by people skilled in the art. As an embodiment of the invention, monoclonal antibody can be prepared from hybridoma cells. For example, expressed known proteins of virus (such as envelop protein) or viral particles are used as antigen to be injected into mice to produce antibody. And screen out hybridoma lines secreting desired monoclonal antibody that can bind specifically to virus by hybridoma technology. The hybridoma cells are cultivated in bioreactors or injected into abdominal cavity of mice to produce high titer antibody quickly in a large amount. The monoclonal antibody produced in the invention can be used directly without purification, which can effectively reduce the production cost.

An embodiment for preparation of phage display antibodies may be performed by the following procedures. First, mRNA fragments are extracted from spleen cells of specific antigen (such as virus antigen) immunized animals or hybridoma lines producing monoclonal antibodies. RT-PCR is carried out to synthesize cDNAs from the mRNA fragments by using a specific primer. The cDNAs are ligated into M13 phages to construct a phage display library, antigens from virus, such as infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), or white spot syndrome virus (WSSV), can be used to screen out phage clones displaying antibodies bound to the antigens of interest.

The selected phage clones can be cultivated and enriched in a host organism system, such organism system can be a bacterial system. Then, phage display antibodies are obtained from the host organism system. And the phage display antibodies can be applied to the compositions of the present invention for reducing virus infection rate in aquatic crustaceans. Production of phage display antibodies does not need serum-containing medium, it is advantageous for simplifying the process and reducing a cost of production.

On the other hand, nucleotides encoding antibodies of interested can be also screened out with the phage display system. Selected nucleotides can be transformed into a host organism system, such as a bacterial or yeast cell, to express the antibodies. The recombinant organism comprising a nucleotide encoding antibody of interested can be cultivated to obtain the antibody in a bioreactor.

To explain the present invention more specifically, embodiments listed below are for preparation of monoclonal antibodies of IHHNV and WSSV. The easily infected WSSV for shrimps is taken as an example. Monoclonal antibodies producing hybridoma line HTWC28 against envelope protein VP28 of WSSV according to the invention can be added directly into clean seawater or mixed with feeds to feed shrimps, in order to effectively control the viral infection of shrimps.

### EXAMPLE 1

(1) Preparation of White Spot Syndrome Virus (WSSV) Antigen

The liver, pancreas and skin are collected and ground after WSSV infected tiger shrimps (Penaeus monodon) are anatomized, which are filtered through a 0.45 μm filter to remove the impurities, followed by ultracentrifugation in a CsCl density gradient (the gradient contains a gradient of 20%, 30%, and 40% CsCl resuspended in 1×TNE buffer containing 20 mM Tris Base, 400 mM NaCl, 5 mM EDTA, pH 7.4) at 39,000 rpm, 4° C. for 18 hours to collect the WSSV viral particles.

The WSSV solution is digested with proteinase K (100 μg/ml) and one-tenth volume of lysis buffer (100 mM Tris-HCl, pH 8.0, 100 mM EDTA, 2.5% SDS), which are incubated at 55° C. for 24 hours. The DNA of WSSV is obtained after phenol chloroform extraction.

And then the known method of polymerase chain reaction (PCR) is performed to amplify the DNA of envelope protein VP28 of WSSV (SEQ ID NO: 1). The DNA of envelope protein VP28 of WSSV comprises 615 nucleotides, which encodes 204 amino acids (SEQ ID NO: 2) and a 28 kDa molecular weight protein.

DNA fragment of PCR amplified products are eluted and purified after agarose gel electrophoresis. This DNA fragment is cloned into pET 28a vectors with ligase. This recombinant plasmid is termed pET28a-VP28.

Plasmid pET28a-VP28 is transformed into E. coli and induced to express VP28 protein. The transformants are cultivated at 37° C. for 3 hours, followed by addition of 0.5 M isopropyl-β-d-thiogalactopyranoside (IPTG) to induce protein expression and cultivated for another 3 hours at 37° C. The VP28 antigen protein is purified by His-tag column chromatography.

(2) Preparation of Infectious Hypodermal Hematopoietic Necrosis Virus, IHHNV antigen

The liver and pancreas are collected and ground after IHHNV infected shrimps (confirmed with PCR reaction) are anatomized, which are filtered through a 0.45 μm filter to remove the impurities, followed by ultracentrifugation in a CsCl density gradient (the gradient contains a gradient of 20%, 30%, and 40% CsCl resuspended in 1×TNE buffer containing 20 mM Tris Base, 400 mM NaCl, 5 mM EDTA, pH 7.4) at 39,000 rpm, 4° C. for 18 hours to collect the IHHNV viral particles.

The IHHNV solution is digested with proteinase K (100 μg/ml) and one-tenth volume of lysis buffer (100 mM Tris-HCl, pH8.0, 100 mM EDTA, 2.5% SDS), which are incubated at 55° C. for 24 hours. The DNA of IHHNV is obtained after phenol/chloroform extraction.

And then the known method of polymerase chain reaction (PCR) is performed to amplify the DNA of envelope protein VP37 of IHHNV (SEQ ID NO: 3). The DNA of envelope protein VP37 of IHHNV comprises 990 nucleotides, which encodes 329 amino acids (SEQ ID NO: 4).

DNA fragment of PCR amplified products are eluted and purified after agarose gel electrophoresis. This DNA fragment is cloned into pET 28a vectors with ligase. This recombinant plasmid is termed pET28a-VP37.

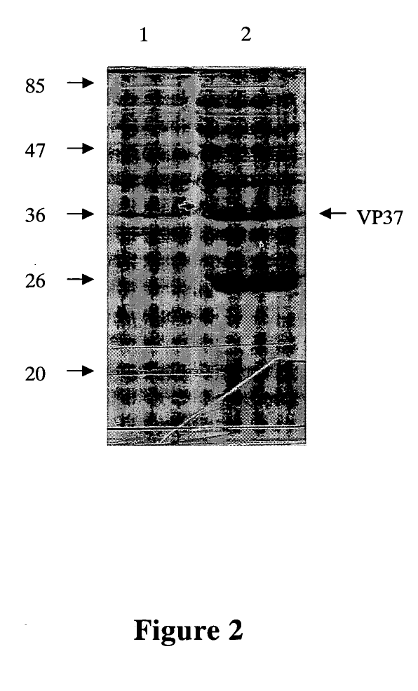

Plasmid pET28a-VP37 is transformed into E. Coli and induced to express VP37 protein. The transformants are cultivated at 37° C. for 3 hours, followed by addition of 0.5 M IPTG to induce protein expression and cultivated for another 3 hours at 37° C., as shown in FIG. 2. The VP37 antigen protein is purified by His-tag column chromatography.

(3) Monoclonal Antibody Preparation

The purified antigen protein is emulsified with Freund's complete adjuvant (FCA). One hundred μg of emulsified antigen is injected into the abdominal cavity of 6-8 week old, healthy BALB/C mouse. Two to three weeks later, emulsification is carried out with Freund's incomplete adjuvant (FIA) and injected into the abdominal cavity of mouse again, and repeated at another two to three weeks later. Blood samples are collected from tail veins of mice after one more week, and the titers are determined with ELISA. Last boost immunization is carried out with 100 μg of purified antigen protein directly injection and cell fusion are carried out after three to four days.

Before cell fusion, the BALB/C mice without immunization are sacrificed after blood sampling, soaked in 75% ethanol for 5 min, and the abdomen is cleaned with iodine. After the mouse abdomen is cut open, 5 ml of HAT select medium containing fetal bovine serum are injected into abdominal cavities. The above solution is aspirated, diluted with another 5 ml of said HAT medium, distributed into a 96-well ELISA plate with one drop per well, and cultivated at 37° C., in a 5% CO₂ incubator overnight.

On the other hand, immunized mice are blood sampled, soaked in 75% ethanol for 5 min. The spleen is removed, and washed with serum-free RPMI1640 medium. Washed spleen is put on a sterile copper net, aspirating several times for the supernatant, and centrifuged at 1500 rpm for 8 min. The pellet is composed of splenocytes ready for use.

The prepared splenocytes from BALB/c mice and SP2/0 myelomas are counted after dilution properly with rinse solution. The cells are mixed in a ratio of 5:1, and centrifuged at 1500 rpm for 8-10 min. The supernatant is removed and 1 ml of 50% polyethylene glycol (PEG) is slowly added and reacted for 1-2 min, followed by slow addition of 20-30 ml of rinse solution. HAT culture medium is added to resuspend cells gently after centrifugation at 1500 rpm for 8-10 min to remove PEG to obtain hybridoma cell suspension.

Newly fused hybridoma is distributed into 96-well tissue culture-treated plates prepared as abovementioned, in one to two drops of suspension per well, and placed in 37° C. incubator containing 5% CO₂. One drop of HAT culture medium is added into each well three days after fusion, and the medium is changed at the fourth day.

ELISA is performed to screen all potential clones from fusion in order to clone hybridoma cells producing anti-VP28 and anti-VP37 monoclonal antibodies.

Purified antigen proteins (VP-28 and VP-37) are diluted with 0.05 M sodium carbonate buffer (0.159% (w/v) sodium carbonate and 0.293% (w/v) sodium bicarbonate, pH 9.6) to 10 μg/ml and added into each well in a 96-well ELISA plate. The plate is covered at 4° C. overnight, and blocked with bovine serum albumin (BSA) at 37° C. for one hour. The antigen solution is dumped out and washed with phosphate buffer (pH 7.4) three times, each time 3-5 min. Then 100 μL of hybridoma cell suspension is added into each well and filled with equal volume of RPMI1640 culture medium, and cultivated at 37° C. for 1-2 hours. The plate is again washed with phosphate buffer (pH 7.4) three times, each time 3-5 min. 100 μl/well of the pre-diluted antimouse IgG enzyme labeled secondary antibody is incubated at 37° C. for 1-2 hours and washed 3 times again. 100 μl/well of OPD-peroxidase substrate is added and incubated at room temperature for 30 min without light. After color development, 50 μl of stop solution (2 M sulfuric acid) is added per well, and the absorbance at 490 nm in an ELISA reader is read. Serum (100-fold dilution) from immunized mice is used as positive control, while serum from myeloma cells is used as a negative control.

Feeder layers are prepared from ascitic fluids of healthy BALB/C mouse. Positive hybridomas are diluted in the concentration of one cell per 100 μl. These diluted hybridoma cells are distributed into 96-well microtiter plates containing with feeder layers 100 μl per well and incubated in 5% CO₂ atmosphere at 37° C. Medium is changed every three days. The antibodies are determined after cell growth for 8-10 days; the positive wells are labeled and changed with fresh medium again. Two days later, the antibodies are determined again. Subcloning of monoclonal lines is repeated twice with wells showing two positive results, till all the wells are positive. The clones with high OD values, high viabilities and single colony formation are selected to amplify. Therefore, hybridoma cell lines producing anti-VP28 antigen and anti-VP37 antigen monoclonal antibodies are obtained.

### EXAMPLE 2

### Titration of Monoclonal Antibodies from Culture Media of Hybridoma Cells

The culture media of monoclonal antibody against WSSV VP-28 antigen protein (termed HTWC thereafter) are diluted in the ratio of 1×10⁻², 2×10⁻², 1×10⁻³, 2×10⁻³ and 1×10⁻⁴ and analyzed with Western blot analysis to confirm the specificity and titer of monoclonal antibody obtained from Example 1.

Protein samples are transferred to nylon membrane after SDS-PAGE analysis in a semi-dry blotter (Panther™ Semidry Electroblotter) at 120 mA for 70 min. The membrane is placed in a blocking buffer (5% non-fat milk powder in TBST (20 mM Tris-HCl, 150 mM NaCl, and 0.05% Tween-20)) for one hour, washed with 25 ml of TBST for 5 min and incubated with 5 ml of blocking buffer containing primary antibody for 2 hours at room temperature. After hybridization, the membrane is washed with 25 ml of TBST for 5 min and incubated with 5 ml of blocking buffer containing secondary antibody for 1 hour at room temperature. The membrane is washed twice with 25 ml of TBST for 10 min twice and washed with TBS (20 mM Tris-HCl, and 150 mM NaCl) 5 min for three times. Then the membrane is developed with 10 ml of APB staining solution containing NBT (Nitro blue tetrazolium) and BCIP (5-Bromo-4-chloro-3-indolyl-phosphate) at room temperature, washed twice with water after color developed.

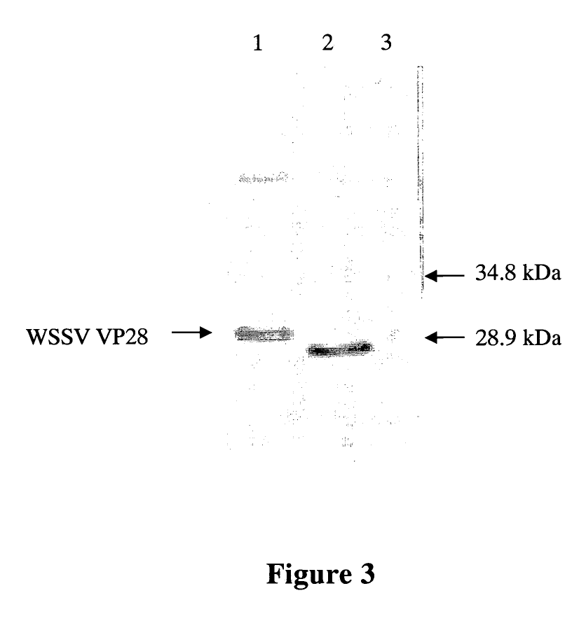

Results shown that HTWC28 can precisely and specifically detect recombinant rVP28 antigen protein and purified VP28 antigen protein of WSSV. On the other hand, the titer determined with Western blot of HTWC produced from hybridoma cell culture is more than 1×10⁴, and the antibodies have high specificity as shown in FIG. 3.

### EXAMPLE 3

Tiger shrimps (Penaeus monodon) each in size of 2.9±0.3 cm, and average weighing approximately of 0.16 g are divided into 3 groups with 15 shrimps in each group. The seawater salinity of the culture pond is adjusted to 16 ppt (1.6%). Each group of shrimps is put into a culture tank with 2 L of seawater and cultivated overnight to accommodate new environment. The extracts of WSSV infected shrimps; monoclonal antibodies against WSSV (HTWC) and TNE buffer are mixed respectively (test solution) for soaking feed diets as indicated in the following Table.

| | Extract of Infected | Monoclonal | |
|---|---|---|---|
| | Shrimps | Antibody | TNE Buffer |
| Group 1 | 125 μl | | 125 μl |
| Group 2 | 125 μl | 125 μl | |
| Group 3 | | 125 μl | 125 μl |

The test solution is mixed thoroughly and stayed at room temperature (around 23-25° C.) for one hour. 0.1 gram of feed diet is added into reacted test solution, mixed and stayed for another hour to absorb test solution.

The water level is adjusted to 0.5 L and the tiger shrimps are hungered for 12 hours before the feeding experiment started. After feed diets are added overnight, the seawater is supplemented to 2 liters followed by the regular cultivation. Two days later, the same treatment is carried out again. The number of surviving shrimps is recorded after the first feeding.

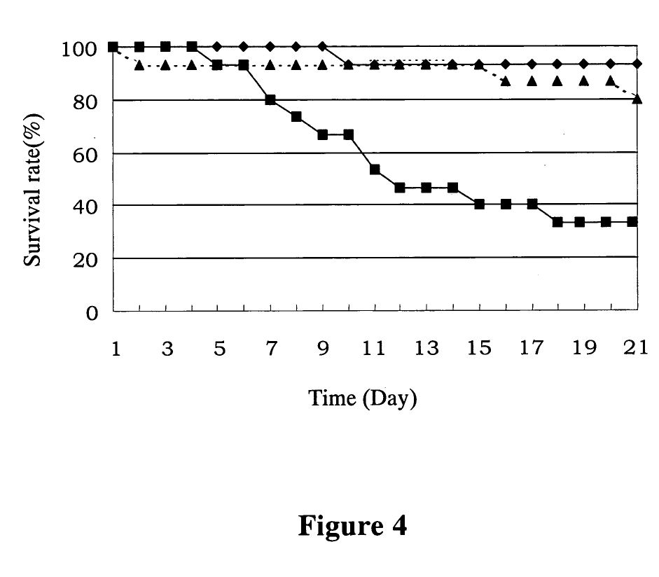

FIG. 4 shows the results after tested for 21 days. Shrimps treated with infected shrimp extract started to die at the fifth day of experiment, and the survival rate was 33% at the 18th day. However, the survival rate of shrimps treated with infected shrimp extract and monoclonal antibody dropped a little bit, but remained around 80-93%. Therefore, the results show that the survival rate is increased 47-60% when WSSV monoclonal antibody is added.

### EXAMPLE 4

Experiments are carried out as described in Example 3 except the diets containing test solution are fed continuously but not twice only. The number of surviving shrimps is recorded after the first feeding.

The experiments are carried out and divided into three groups depending upon the different growth stages of shrimps. Group 1 is each 3.92±0.28 cm in length, and on average 0.45 g in weight; Group 2 is each 3.32±0.32 cm in length, and on average 0.25 g in weight; and Group 3 is each 2.48±0.29 cm in length, and on average 0.15 g in weight. The diets containing test solution are used to feed the shrimps. The number of surviving shrimps is recorded after the first feeding.

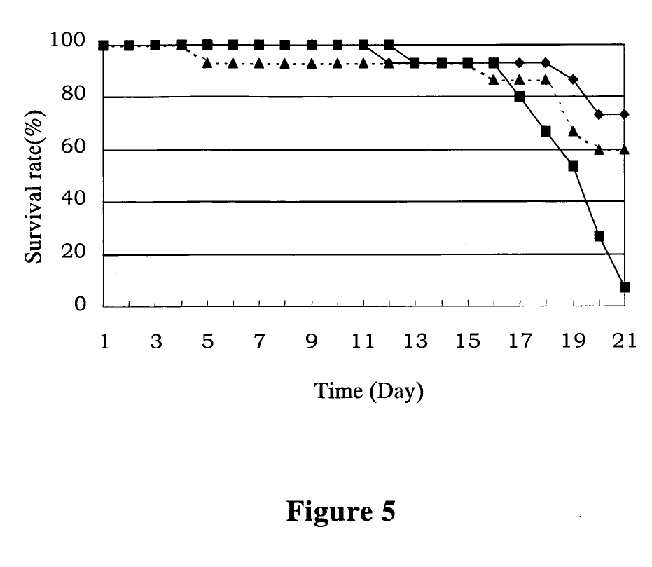

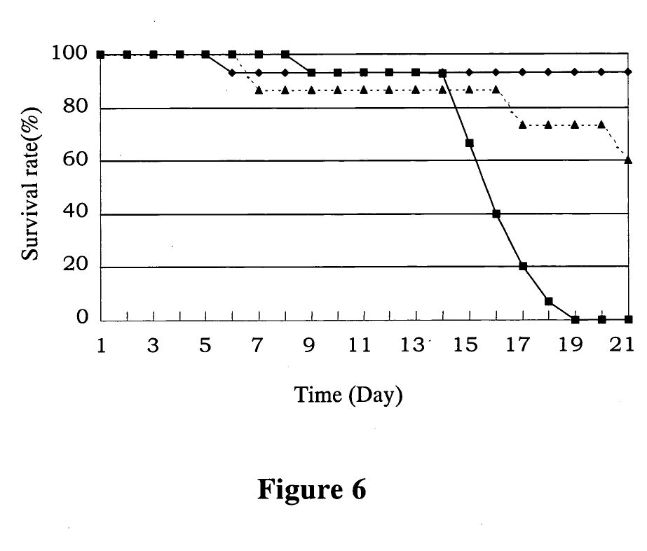

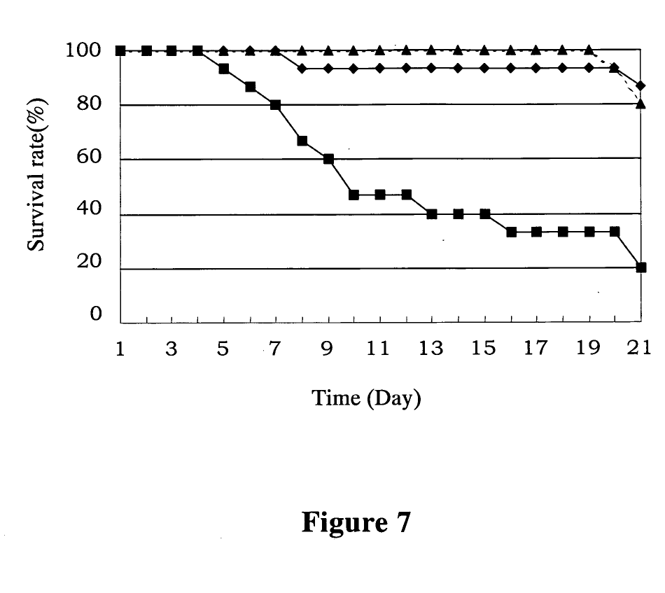

The results are recorded for 21 days and show in FIG. 5, FIG. 6 and FIG. 7. These three experiment groups are termed large or medium or small-size shrimp for easy explanation according to their sizes. FIG. 5 indicates the result of large-size shrimp group, FIG. 6 medium-size and FIG. 7 small-size shrimp group. FIG. 5 shows that large-size shrimps fed with infected extracts start to die remarkably after 16 days of experiment; the survival rate reached 6.7% at 21 day. The survival rates of shrimps fed with antibodies or antibodies and infected extracts also dropped after 18 days of experiment, but still showed a survival rate of 60% and 73.3%, respectively. Therefore, the survival rates increases 53-66% if antibodies are supplemented.

Medium-size shrimps fed with infected extracts start to die remarkably after 14 days of experiment; the survival rate reached 0% at 19 day. The survival rates of shrimps fed with antibodies and infected extracts also dropped after 18 days of experiment, but stopped at a survival rate of 60%. And shrimps fed with antibodies show a survival rate of 90% till the end of experiment. Therefore, the survival rates increases 60-90% if antibodies are supplemented.

Small-size shrimps fed with infected extracts start to die continuously after 4 days of experiment; the survival rate reached 20% at 21 day. The survival rates of shrimps fed with antibodies or antibodies and infected extracts still show a survival rate more than 80%. Therefore, the survival rates increases 60% if antibodies are supplemented.

In summary, no matter what experiments are carried out, the shrimps fed with infected extracts as diets will continuously and remarkably die, result in the survival rate to less than 30%. But the survival rate can be increased 47% to 60% on average by adding monoclonal antibodies of the present invention.

The in vivo experiments show that monoclonal antibodies of the present invention can bind virus successfully and inhibit virus persistently infecting shrimps, and increase the survival rate of shrimps to above 47%.

### EXAMPLE 5

To determine the effects of HTWC antibody treatment on virus infected shrimps, shrimps in size of 2.16±0.28 cm, and 0.14 g in weight are used for experiments. Eleven shrimps are included in each group, and cultivated in 2 L of breeding water.

Each group is fed morning and night with infected extracts soaked diet for one day. The soaked diet is prepared by adding 250 μl of infected extract to 0.1 g of diet and soaking for one hour at room temperature. Next day is the first day of treating experiment. Infected shrimps are divided into four groups, fed with pre-soaked HTWC diets in 100-fold titer twice at day 1, day 3, day 5 and day 7. The pre-soaked HTWC diets is prepared by adding 250 μl of HTWC to 0.1 g of diet and soaking for one hour at room temperature. Regular diets without treatment are fed unless in the period of experiment. The experiment period is 21 days. The survival rates of shrimps are observed and recorded.

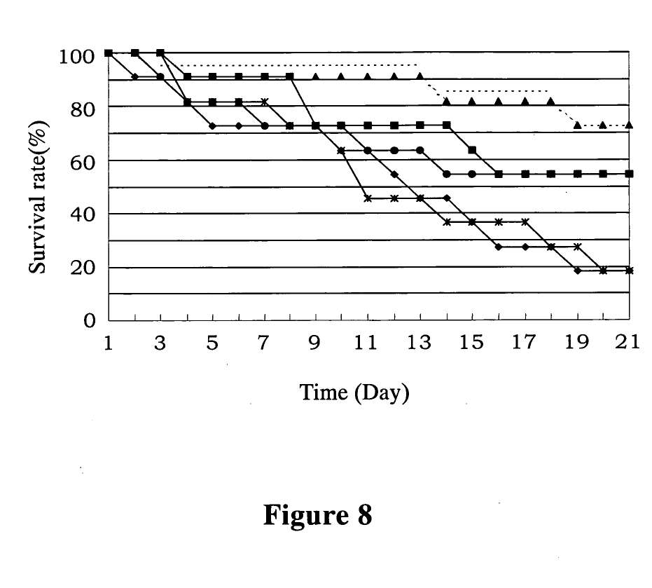

FIG. 8 shows that the shrimps fed with infected extracts but no HTWC soaked diet start to die continuously at the 5th day of experiment, the survival rate drops to 18.2% at the 21st day. The survival rates of shrimps fed with HTWC at the 7th day are similar to the aforementioned group (drops to 18.2% at the 21st day). Those fed HTWC at the 3rd and 5th day show a survival rate of 54.5% at the 21st day, and the former had a slower mortality than the latter. Shrimps fed HTWC at the first day of infection show a survival rate of 90.9% at the 12th day, and 72.7% at the 21st day. Therefore, the survival rates of shrimps increase 54.5% if HTWC is supplemented at the first day of infection, and the rates can increase 36.3% if HTWC is supplemented at the 3rd and the 5th day. The HTWC treatment shows good effect toward virus infection and the timing for treatment is also important, the early the better, precisely, with better survival rate.

Though the present invention is explained in the above embodiment, the present invention disclosed above is not limited by these examples. The present invention may be altered or modified and all such variations are within the scope and spirit of the present invention.

1. A composition for reducing virus infection rates in crustaceans, comprising at least one of the monoclonal antibodies that can bind specifically to virus, the monoclonal antibodies are prepared in the steps of:
(1) obtaining a specific antigenic protein of the virus or virus particles;
(2) using the specific antigenic protein or virus particles to screen out hybridoma lines producing monoclonal antibodies specific to the specific antigen protein by hybridoma technology; and
(3) producing monoclonal antibodies specific to the virus from the hybridoma lines.

2. The composition as claimed in claim 1, wherein the crustaceans are shrimps or crabs.

3. The composition as claimed in claim 1, wherein the virus is selected from the group consisting of infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), and white spot syndrome virus (WSSV).

4. The composition as claimed in claim 1, wherein the virus is infectious hypodermal hematopoietic necrosis virus (IHHNV).

5. The composition as claimed in claim 4, wherein the specific antigenic protein comprises a sequence as SEQ ID No: 4.

6. The composition as claimed in claim 1, wherein the virus is white spot syndrome virus (WSSV).

7. The composition as claimed in claim 6, wherein the specific antigenic protein comprises a sequence as SEQ ID No: 2.

8. The composition as claimed in claim 1, wherein step (2) comprises performing cell fusion using SP2/0 myelomas.

9. The composition as claimed in claim 1, wherein step (2) comprises screening out the hybridoma lines with ELISA method.

10. The composition as claimed in claim 1, wherein step (3) comprises producing the monoclonal antibodies in a large scale from hybridoma lines by a bioreactor.

11. The composition as claimed in claim 1, wherein step (3) comprises producing the monoclonal antibodies by injecting hybridoma into the abdominal cavity of mammals to induce tumor formation, followed by harvesting monoclonal antibodies from ascitic fluids.

12. The composition as claimed in claim 11, wherein the mammal is mouse.

13. A method for reducing virus infection rates in shrimps, comprising the step of administrating a composition, which comprises at least one of the antibodies that can bind specifically to the virus and is mixed with a medium, to the shrimps in specified growth stages, and the antibody is selected from the group consisting of monoclonal antibody, phage display antibody and antibody produced by a recombinant organism.

14. The method as claimed in claim 13, wherein the specified growth stages are selected from the group consisting of gravid shrimp stage, fertilized-egg stage, and nauplii stage.

15. The method as claimed in claim 14, wherein the medium is breeding water.

16. The method as claimed as claim 13, wherein the specified growth stages are selected from the group consisting of zoeal stage, mysis stage and postlarvae stage.

17. The method as claimed in claim 16, wherein the medium is breeding water or feed.

18. The method as claimed in claim 13, wherein the specified growth stages are selected from the group consisting of juvenile stage and adult stage.

19. The method as claimed in claim 18, wherein the medium is feed.

20. The method as claimed in claim 13, wherein the virus is selected from the group consisting of infectious hypodermal hematopoietic necrosis virus (IHHNV), baculovirus penaei (BP), baculoviral midgut GI and necrosis virus (BMN), monodon baculovirus (MBV), hepatopancreatic parvo-like virus (HPV), reo-like virus, Taura syndrome virus, yellow head virus (YHV), and white spot syndrome virus (WSSV).

21. The method as claimed in claim 13, wherein the virus is white spot syndrome virus.

22. A medical composition for reducing virus infection rates in crustaceans, which comprises an effective dosage of composition comprising at least one of the antibodies that can bind specifically to the virus and a pharmacologically acceptable carrier, and the antibody is selected from the group consisting of monoclonal antibody, phage display antibody and antibody produced by a recombinant organism.

23. A feed composition for reducing virus infection rates in crustaceans, which comprises the composition comprising at least one of the antibodies that can bind specifically to the virus, and the antibody is selected from the group consisting of monoclonal antibody, phage display antibody and antibody produced by a recombinant organism.

24. A nutrition supplement composition for reducing virus infection rates in crustaceans, which comprises the composition comprising at least one of the antibodies that can bind specifically to the virus, and the antibody is selected from the group consisting of monoclonal antibody, phage display antibody and antibody produced by a recombinant organism.
